(19) United States
(12) Patent Application Publication
(10) Pub. No.: US 2024/0139469 A1
(43) Pub. Date: May 2, 2024

(54) MINIMALLY INVASIVE INTERVENTIONAL DEVICE AND MINIMALLY INVASIVE INTERVENTIONAL APPARATUS INCLUDING MINIMALLY INVASIVE INTERVENTIONAL DEVICE

(71) Applicant: PEKING UNIVERSITY, Beijing (CN)

(72) Inventors: Xiaoyu Su, Beijing (CN); Tianyun Huang, Beijing (CN); Huiling Duan, Beijing (CN)

(21) Appl. No.: 18/499,063

(22) Filed: Oct. 31, 2023

(30) Foreign Application Priority Data
Nov. 1, 2022 (CN) 202211356126.3
Jan. 31, 2023 (CN) 202310118777.7

(51) Int. Cl. A61M 25/00; A61L 29/02

(52) U.S. Cl. CPC A61M 25/0045; A61L 29/02; A61M 2039/1022

(57) ABSTRACT

The present disclosure relates to a minimally invasive intervention device and a minimally invasive intervention apparatus including the minimally invasive intervention device. Minimally invasive intervention devices includes an inner layer, an intermediate layer, and an outer layer, the intermediate layer covers an outer peripheral surface of the inner layer, and the outer layer covers an outer peripheral surface of the intermediate layer; and the intermediate layer includes a plurality of line bodies, at least some of the line bodies can transmit signals and/or electric energy. The minimally invasive intervention device in the present application has signal transmission capability and high degree of integration, and when the microcatheter or the micro-guide wire is applied in the minimally invasive intervention apparatus, the functional integration and clinical practicality of the minimally invasive intervention apparatus can be improved, thereby reducing the workload of medical personnel in minimally invasive intervention surgery.

### CROSS REFERENCE TO RELATED APPLICATIONS

The present application claims priority to Chinese Patent Application No. 202211356126.3 tilted “MICROCATHETER AND MINIMALLY INVASIVE INTERVENTION APPARATUS” and filed on Nov. 1, 2022, and to Chinese Patent Application No. 202310118777.7 titled “MICRO-GUIDE WIRE AND MINIMALLY INVASIVE INTERVENTION APPARATUS” and filed on Jan. 31, 2023, both of which are hereby incorporated by reference in their entireties.

### TECHNICAL FIELD

The present application relates to the technical field of medical device, specifically to a minimally invasive intervention device and a minimally invasive intervention apparatus including the minimally invasive intervention device, where the minimally invasive intervention device can be, for example, a microcatheter, or a micro-guide wire.

### BACKGROUND

Minimally invasive intervention technology has advantages of being fast, accurate, effective, easy to recover, and difficult to recur. In recent years, it has gradually developed into one of the three major diagnostic and treatment technologies alongside internal medicine and surgery. When using minimally invasive intervention technology for diagnosis and treatment, it is usually necessary to use a micro-guide wire and a microcatheter to deliver drugs or micro-instruments into a focus area to achieve precise treatment.

In clinical applications, when using the micro-guide wire and the microcatheter to transport drugs or micro-instruments, other auxiliary means are generally needed to cooperate with them, by, for example, obtaining real-time position information of the microcatheter through visual feedback, obtaining changes in the human environment in an area where the microcatheter is located by a force sensor, or performing electrical stimulation operations on the focus area; however, currently, the microcatheter generally does not have extensible information acquisition and operation functions, mainly because the microcatheter lack electricity transmission and signal transmission capabilities, and thus the practicality of the microcatheter is seriously limited, bringing inconvenience to the diagnosis and treatment process and increasing the burden on medical personnel. In addition, the existing micro-guide wires only have guiding function, and positioning of the end portions of the micro-guide wires needs the help of angiography apparatus; further, the micro-guide wires cannot be integrated with more sensors, which to some extent limit the intelligent level of vascular intervention surgery and put forward high skill and experience requirements for medical personnel.

Therefore, it is necessary to enhance the practicality of the microcatheter and micro-guide wire to reduce the burden on medical personnel.

### SUMMARY

The present application provides a minimally invasive intervention device and a minimally invasive intervention apparatus including the minimally invasive intervention device, the minimally invasive intervention device can be for example, a microcatheter, or a micro-guide wire, and the purpose of the present application is to improve the practicality of the microcatheter and enrich the functions of the micro-guide wire, so as to simplify the overall structure of the minimally invasive intervention apparatus, reduce the operational difficulty for medical personnel in minimally invasive intervention surgery, and reduce the burden on the medical personnel.

On a first aspect, the present application provides a minimally invasive intervention device, including: an inner layer, an intermediate layer, and an outer layer, the intermediate layer covers an outer peripheral surface of the inner layer, and the outer layer covers an outer peripheral surface of the intermediate layer; and the intermediate layer includes a plurality of line bodies, at least some of the line bodies can transmit signals and/or electric energy.

In some embodiments, the plurality of line bodies are formed as a plurality of conducting wires, and the plurality of conducting wires are adapted for transmitting signals and/or transmitting electric energy.

In some embodiments, each of the conducting wires includes: a core body adapted for transmitting signals and/or transmitting electric energy; an isolation layer arranged on an outer side of the core body, and adapted to isolate the signals from external signals; and an insulation layer arranged between the core body and the isolation layer, and adapted to insulate and isolate the core body from the isolation layer.

In some embodiments, the isolation layer includes a plurality of metal wires arranged in a crossing manner.

In some embodiments, the core body is made of metal or alloy material, the isolation layer is made of conductive material, and the insulation layer is made of insulating material.

In some embodiments, the core body is formed as a copper core, the copper core has a diameter A, and the metal wire has a diameter B, where 1≤A/B≤20.

In some embodiments, the core body is formed as a copper core, the copper core has a diameter A, and the insulation layer has an outer diameter C, where 0.1≤A/C≤10.

In some embodiments, the inner layer is formed as an inner tube layer, which includes a cavity with openings at two ends, the intermediate layer is formed by winding the plurality of line bodies, and the intermediate layer is formed as a spiral or mesh structure.

In some embodiments, the minimally invasive intervention device further includes a coating coated on an inner wall of the inner tube layer.

In some embodiments, the inner layer is formed as an inner core, and the inner core and the intermediate layer form a functional core; and the functional core extends in a first direction and includes a first end, the outer layer covers an outer peripheral surface of a portion of the functional core other than the first end, and the first end can enter a focus area.

In some embodiments, each line body is arranged spirally around an outer peripheral surface of the inner core, and in a cross-section perpendicular to the first direction, the plurality of line bodies are arranged along a circumferential direction of the inner core; or, the respective line bodies are crossed by each other to form a mesh structure.

In some embodiments, the line bodies include a plurality of support lines and conducting wires with the number of 2N, N being a positive integer, the conducting wires can transmit signals and/or electric energy, and each conducting wire forms a transmission loop with another conducting wire.

In some embodiments, the intermediate layer is formed with a plurality of transmission loops, some of the transmission loops are adapted for transmitting electric energy, and others of the transmission loops are adapted for transmitting signals.

In some embodiments, the minimally invasive intervention device includes an end portion and an extension portion, a portion of the functional core not covered by the outer layer forms the end portion, and the portion of the functional core covered by the outer layer and the outer layer form the extension portion; in a cross-section perpendicular to the first direction, a radial dimension of the extension portion gradually decreases in a direction from the extension portion to the end portion.

In some embodiments, the extension portion includes a support portion, a transition portion and a guide portion connected to each other in the direction from the extension portion to the end portion; the support portion is formed in a cylindrical shape; and radial dimensions of the transition portion and the guide portion both gradually decrease in the direction from the extension portion to the end portion.

On a second aspect, the present application provides a minimally invasive intervention apparatus, including: the minimally invasive intervention device as any of above embodiments; a signal transceiver/power supply connected to one end of the minimally invasive intervention device; and an actuator/sensor connected to an end of the minimally invasive intervention device away from the signal transceiver/power supply.

On a third aspect, the present application proposes a minimally invasive intervention apparatus, including a sensing/executing element, a control/computing/power supply element, and the minimally invasive intervention device as any of the above embodiments, wherein the intermediate layer of the minimally invasive intervention device is electrically connected to the sensing/executing element at a first end of the line bodies, and an end of the intermediate layer covered by the outer layer is electrically connected to the control/computing/power supply element.

According to the embodiments of the present application, the microcatheter includes the inner tube layer, the intermediate layer and the outer tube layer, the inner tube layer includes a cavity with openings at two ends, and the intermediate layer is arranged on the outer wall of the inner tube layer, the intermediate layer is formed by winding a plurality of line bodies, and at least some of the plurality of line bodies are adapted for transmitting signals; the outer tube layer covers the intermediate layer. As such, at least some of the line bodies for forming the intermediate layer can transmit signals, and thus the microcatheter itself has signal transmission capability; when the microcatheter is applied in the minimally invasive intervention apparatus, it can improve the practicality of the minimally invasive intervention apparatus in clinical practice and reduce the workload of medical personnel in minimally invasive intervention surgery. Moreover, since the intermediate layer with signal transmission function is integrated into the microcatheter itself, the microcatheter has a high degree of integration, which can minimize the working diameter of the microcatheter and meet clinical requirements in more situations, further expanding the application scenarios of the microcatheter.

According to the embodiments of the present application of the micro-guide wire and the minimally invasive intervention apparatus including the micro-guide wire, by providing the functional core with the functional layer and enabling at least some line bodies of the functional layer to transmit signals and/or electric energy, the elements for position detection can be directly installed to the first end of the functional core and connected with the line bodies for transmitting signals and/or electric energy, and the line bodies for transmitting signals and/or electric energy can replace functional components used for data transmission in existing technology; as such, the functions of the micro-guide wire can be enriched, the overall structure of the minimally invasive intervention apparatus can be simplified, and the operational difficulty for the medical personnel during use can be reduced.

### BRIEF DESCRIPTION OF THE DRAWINGS

Features, advantages, and technical effects of exemplary embodiments of the present disclosure will be described below with reference to the accompanying drawings.

FIG. 1 is a schematic diagram of principle of a minimally invasive intervention apparatus provided in some embodiments of the present application;

FIG. 2 is a schematic diagram of partial structure of a microcatheter provided in some embodiments of the present application;

FIG. 3 is a schematic diagram of partial structure of wires in an intermediate layer of a microcatheter provided in some embodiments of the present application;

FIG. 4 is a schematic diagram of a structure of a micro-guide wire provided in some embodiments of the present application;

FIG. 5 is a schematic diagram of partial structure of a micro-guide wire of another embodiment of the present application;

FIG. 6 is a schematic diagram of partial structure of a conducting wire provided in some embodiments of the present application.

The accompanying drawings may not necessarily be drawn to actual proportions.

### DESCRIPTION OF REFERENCE NUMERALS

1, microcatheter;
10, inner tube layer; 11, cavity;
20, intermediate layer; 21, wire; 211, core body; 212, insulation layer; 213, solation layer;
30, outer tube layer;
2, signal transceiver; 3, actuator;
1′, functional core; 11′, inner core; 12′, functional layer; 121′, conducting wire; 1211′, core line; 1212′, insulation layer; 1213′, shielding layer; 122′, transmission loop; 2′, covering layer; 3′, end portion; 4′, extension portion; 41′, support portion; 42′, transition portion; 43′, guide portion; X, first direction.

### DETAILED DESCRIPTION

Below, embodiments of the present disclosure will be further described in detail with reference to the accompanying drawings and embodiments. The detailed description of the embodiments and the accompanying drawings are intended to exemplarily illustrate the principles of the present disclosure and are not intended to limit the scope of the present disclosure. That is, the present disclosure is not limited to the described embodiments.

In the description of the present disclosure, it should be noted that, unless otherwise stated, the meaning of “a plurality of” is two or more; the orientations or positional relationships indicated by the terms “upper”, “lower”, “left”, “right”, “inner”, “outer” and the like are merely for the purpose of describing the present disclosure and simplifying the description, and are not intended to indicate or imply that the device or component referred to has a particular orientation, is constructed and operated in a particular orientation, and therefore cannot be understood as a limitation of the present disclosure. Moreover, the terms “first”, “second” and the like are used for descriptive purposes only and are not to be construed as indicating or implying relative importance. “Vertical” is not strictly vertical, but within an allowable range of error. “Parallel” is not strictly parallel, but within an allowable range of error.

The directional words appearing in the following description are all the directions shown in the drawings and do not limit the specific structure of the present application. In the description of the present application, it should also be noted that unless otherwise specified and limited, the terms “installation”, “connection”, and “connection” should be broadly understood, for example, they can be fixed connections, detachable connections, or integrated connections; they can be direct connections or indirect connections through intermediate media. For person skilled in the art, the specific meanings of the above terms in the present application can be understood according to specific circumstances.

Minimally invasive intervention technology has advantages of being fast, accurate, effective, easy to recover, and difficult to recur, and in recent years, it has gradually developed into one of three major diagnostic and treatment technologies alongside internal medicine and surgery. When using the minimally invasive intervention technology for diagnosis and treatment, a cooperation of the microcatheter and the micro-guide wire is generally needed, where the micro-guide wire is placed inside the microcatheter and can slide along the microcatheter, and after a location of a focus area is determined by using the micro-guide wire, the micro-guide wire is drawn out, and drugs or micro therapeutic instruments are delivered to the focus area by using the microcatheter; in general, the guide wire is first, followed by the catheter. In clinical applications, surgical requirements cannot be fully met merely by relying on the microcatheter and the micro-guide wire, and for example, in order to improve the positioning accuracy of the focus area, it is generally necessary to continuously shoot the microcatheter and micro-guide wire with X-rays to obtain real-time positions of the microcatheter and micro-guide wire; however, these additional auxiliary means make the surgical process more cumbersome and increase the workload of medical personnel.

The inventors found that the microcatheter generally is formed in a three-layer structure, where an inner layer is a tube with a cavity and a lubricated surface to facilitate delivery of substances within the catheter, an intermediate layer is made of metal (generally stainless steel) braided mesh or spring coil, which serves as a support and reinforcement to prevent the catheter from breaking or plastic deformation after repeated bending, and an outer layer is wrapped by soft materials such as silicone and resin. In manufacturing, these three layers are generally manufactured separately, and finally nested and combined together in an ultra-clean space by hot melting. However, the microcatheter itself only has the function of substance delivery and does not have extensible information acquisition and operation functions, mainly because it lacks power supply and signal transmission capabilities; thus, the practicality of the microcatheter is relatively limited, which cannot reduce the burden on medical personnel in clinical applications.

In view of the above issues found by the inventors, the inventors improve the structure of the microcatheter and propose a microcatheter, which includes an inner tube layer, an intermediate layer, and an outer tube layer; the inner tube layer includes a cavity with openings at two ends, the intermediate layer is arranged on an outer wall of the inner tube layer, and is formed by winding a plurality of line bodies, at least some of the line bodies are adapted for transmitting signals, and the outer tube layer covers the intermediate layer. As such, at least some of the line bodies for forming the intermediate layer can transmit signals, and thus the microcatheter itself has signal transmission capability, which improves the practicality of the microcatheter. Moreover, since the intermediate layer with signal transmission function is integrated into the microcatheter itself, the microcatheter has a high degree of integration, which can minimize the working diameter of the microcatheter and thus meet clinical requirements in more situations, further expanding the application scenarios of the microcatheter.

Meanwhile, the inventors further propose a minimally invasive intervention apparatus, which includes the microcatheter with signal transmission capability, as well as a signal transceiver and an actuator, the signal transceiver is adapted to emit and receive signals, and the actuator is adapted to receive and execute instructions from the signal transceiver. In clinical applications, the minimally invasive intervention apparatus in the present application has multiple functions, can provide operational convenience and reduce work burden for medical personnel in the minimally invasive intervention surgery. Moreover, due to the high integration of the microcatheter, the working diameter of the microcatheter can be minimized to the greatest extent, thereby further expanding the application scenarios of the minimally invasive intervention apparatus.

The embodiments of the present application will be further described in detail below in combination with the accompanying drawings.

FIG. 1 is a schematic diagram showing principle of a minimally invasive intervention apparatus provided in some embodiments of the present application.

As shown in FIG. 1, the minimally invasive intervention apparatus includes a microcatheter 1, a signal transceiver 2, and an actuator 3, the signal transceiver 2 is connected to one end of the microcatheter 1, and the actuator 3 is connected to an end of the microcatheter 1 away from the signal transceiver 2.

The microcatheter 1 not only has substance delivery function, but also has signal transmission capability. The microcatheter 1 can be transformed into various forms according to different usage requirements, such as being curved or bent under external forces.

The signal transceiver 2 can emit and receive signals, and it can be an independent device or a collection of multiple connected devices. The signal transceiver 2 can further have control functions, such as being a controller with signal emission and reception capabilities. The signal transceiver 2 itself can further have displaying functions, such as being provided with a display panel, or the signal transceiver 2 can be connected to an external display device. The signal transceiver 2 can be provided with a power supply device, such as a battery, or it can be connected to an external power device.

The actuator 3 can be formed in various structures, such as a probe with force sensing function, a camera with image sensing function, or a micro-robot with surgical operation function. The actuator 3 can be one or more, and in case of more than one actuators 3, the functions of the more than one actuators 3 can be the same or different. Alternatively, one actuator 3 can be integrated with a plurality of functions simultaneously, which can further enhance the versatility of the minimally invasive intervention apparatus in clinical applications and thus enhance the practicality thereof. In clinical applications, the actuator 3 can be configured with various functional modes according to different application scenarios of the minimally invasive intervention surgery, and the present application does not limit this aspect.

It should be noted that the signal referred to in the embodiments of the present application can be an electrical signal, an optical signal, etc.

The minimally invasive intervention apparatus including the microcatheter in the present application has multifunctional applications and can replace some auxiliary medical means in the minimally invasive intervention surgery in clinical applications, thereby providing convenience for operation of the minimally invasive intervention surgery, reducing difficulty of the surgery, and reducing the burden on medical personnel. Moreover, the microcatheter of the minimally invasive intervention apparatus in the present application has high degree of integration, which can minimize the working diameter of the microcatheter to the greatest extent and expand the application scenarios of the minimally invasive intervention apparatus.

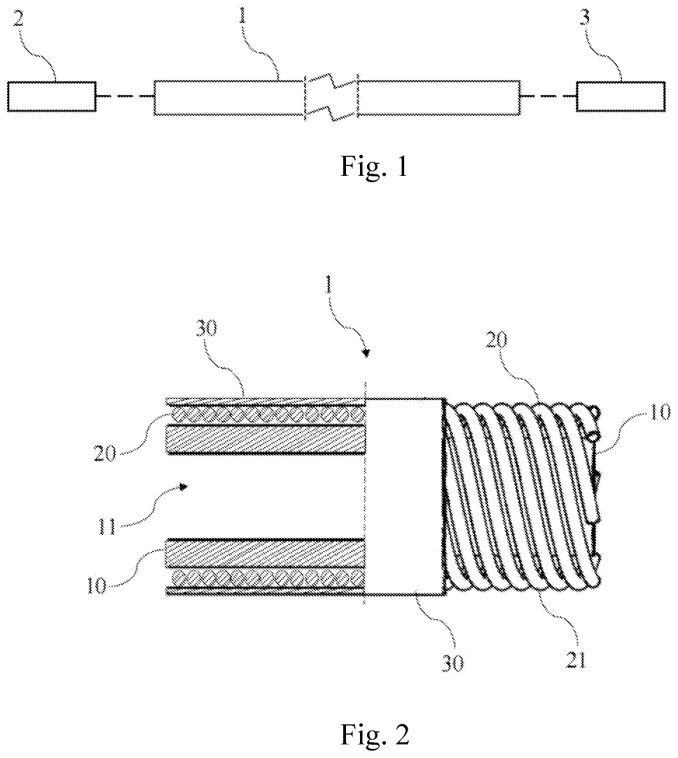

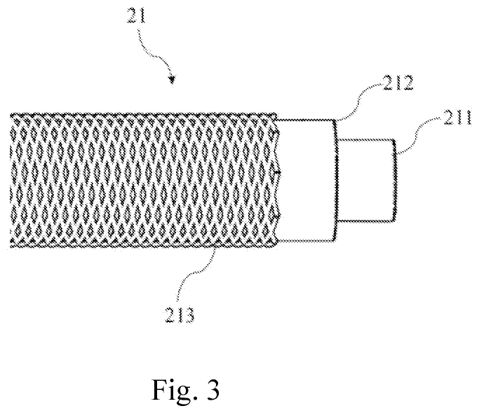

FIG. 2 is a schematic diagram of a partial structure of the microcatheter provided in some embodiments of the present application; FIG. 3 is a schematic diagram of a partial structure of a wire in an intermediate layer of the microcatheter provided in some embodiments of the present application.

As shown in FIGS. 1 to 3, in some embodiments, the microcatheter 1 includes an inner tube layer 10, an intermediate layer 20, and an outer tube layer 30; the inner tube layer 10 includes a cavity 11 with openings at two ends; the intermediate layer 20 is arranged on an outer wall of the inner tube layer 10 and is formed by winding a plurality of wires 21, and at least some of the plurality of wires 21 are adapted for transmitting signals; the outer tube layer 30 covers an outer peripheral surface of the intermediate layer 20.

The inner tube layer 10 is a tube structure with a cavity, and in order to facilitate the transportation of substances in the cavity 11, the inner tube layer 10 can be made of materials with high lubricity, such as PTFE (Poly Tetra Fluoroethylene). The inner tube layer 10 can be a tubular structure in various shapes, such as a cylindrical tubular structure or a square tubular structure. The cavity 11 may be formed in various shapes, and for example, the cavity 11 may be a cylindrical cavity or a square cavity. Exemplarily, in FIG. 2, the inner tube layer 10 is a cylindrical tubular structure, and the cavity 11 is a cylindrical cavity.

The intermediate layer 20 is arranged on the outer wall of the inner tube layer 10, and plays a certain supporting and reinforcing role on the microcatheter 1. The intermediate layer 20 can be formed in various structural forms, such as a mesh structure or a spiral structure. For example, in FIG. 2, the intermediate layer 20 is formed in a spiral structure.

The intermediate layer 20 is formed by winding the plurality of wires 21, and at least some of the wires 21 are adapted for signal transmission, that is, it can be understood that, the plurality of wires 21 are all adapted for signal transmission, or, only some of the wires 21 are adapted for signal transmission, while other wires can have electricity transmission function, or, some of the wires 21 have neither signal transmission capability nor electricity transmission capability, while can only have function of support and reinforcement. According to the types of transmitted signals, the wire 21 can be formed in various structures; for example, when the transmitted signal is an optical signal, the wire 21 can be a light-guide fiber; alternatively, when the transmitted signal is an electrical signal, the wire 21 can be an electric wire.

The outer tube layer 30 covers the outer peripheral surface of the intermediate layer 20, and the outer tube layer 30 can protect the intermediate layer 20 and the inner tube layer 10. The outer tube layer 30 can be made of various materials, such as silicone, resin, etc.

According to the embodiments of the present application, the microcatheter 1 includes an inner tube layer 10, an intermediate layer 20, and an outer tube layer 30, the intermediate layer 20 is formed by winding a plurality of wires 21, with at least some of the plurality of wires 21 adapted for transmitting signals, and the outer tube layer 30 covers the intermediate layer 20. As such, at least some of the wires 21 for forming the intermediate layer 20 can transmit signals, and thus the microcatheter 1 itself has signal transmission capability, which can improve the practicality of the microcatheter 1. When the microcatheter 1 is applied to the minimally invasive intervention apparatus, multiple sensing and execution functions in clinical applications can be provided by the microcatheter 1, thereby improving the operational convenience of the minimally invasive intervention surgery, reducing difficulty of the surgery, and reducing the burden on medical personnel.

In some embodiments, the plurality of wires 21 are adapted to transmit electrical signals and transmit electric energy. The plurality of wires 21 can be formed in multiple different implementation forms.

In some examples, some of the plurality of wires 21 are adapted to transmit electrical signals, while some other wires are adapted to transmit electric energy.

In some other examples, some of the plurality of wires 21 are adapted to transmit electrical signals, some of the wires are adapted to transmit electric energy, and some others of the wires are only adapted to support and reinforce the microcatheter 1, which can support the wall of the outer tube layer to maintain its shape, protect the inner tube layer from being compressed, and can also transmit forces and moments generated by the surgeon's hand operation in the minimally invasive intervention surgery.

In some other examples, at least some of the plurality of wires 21 have both electrical signal transmission and electric energy transmission functions.

In some examples, the plurality of wires 21 can all serve to support the morphology of the wall of the outer tube layer.

As such, the plurality of wires 21 can not only transmit electrical signals, but also can transmit electric energy, which further improve the functional practicality and integration of the microcatheter 1.

Exemplarily, in the embodiment of the present application, the number of wires 21 can be 6.

As shown in FIGS. 1 to 3, in some embodiments, the wire 21 includes a core body 211, an isolation layer 213, and an insulation layer 212; the core body 211 is adapted to transmit electrical signals and/or transmit electric energy; the isolation layer 213 is arranged on an outer side of the core body 211, and is adapted to isolate electrical signals from external signals; the insulation layer 212 is arranged between the core body 211 and the isolation layer 213, and is adapted to insulate and isolate the core body 211 from the isolation layer 213.

The core body 211 is adapted to transmit electrical signals and/or transmit electric energy, meaning that the core bodies 211 of some of the plurality of wires 21 are adapted to transmit electrical signals and the core bodies 211 of some other of the plurality of wires 21 are adapted to transmit electric energy, or that the core bodies 211 of some of the plurality of the wires 21 have both electrical signal transmission and electric energy transmission capabilities. The core body 211 is formed by a conductor material, and it can be a metal material, such as copper, aluminum, etc., or, it can be a non-metallic material, such as graphite.

The isolation layer 213 is located on an outer side of the core body 211 and is adapted to isolate the electrical signals from external signals, meaning that the isolation layer 213 has anti-signal interference function. The isolation layer 213 can be formed in various structural forms, such as a metal braided layer in a mesh shape or a metal coating layer.

The insulation layer 212 is arranged between the core body 211 and the isolation layer 213, and the insulation layer 212 can be made of various materials, such as PP (polypropylene) or PE (polyethylene).

By providing the wire 21 including the core body 211, the isolation layer 213 and the insulation layer 212, while ensuring the electric conductivity and signal transmission capability of the wire 21, the anti-interference capability of the wire 21 can also be improved, thereby improving the stability of the signal transmission of the wire 21.

In some embodiments, the core body 211 is formed as a copper core. Copper has a low resistivity and balanced conduction for full frequency, which can improve the electric conductivity of the wire 21, and by using copper to make the core body 211, the manufacturing process is easier with relatively low production costs.

As shown in FIGS. 1 to 3, in some embodiments, the isolation layer 213 includes a plurality of metal wires, which are arranged in a crossing manner.

A plurality of metal wires are crossed by each other to form the isolation layer 213, and it can also be considered as that the isolation layer 213 is formed in a mesh structure, as shown in FIG. 3. The metal wire can be made of various materials, such as red copper or tinned copper. Exemplarily, in the embodiment of the present application, the metal wire can also be made of iron nickel alloy.

By forming the isolation layer 213 with a plurality of metal wires crossing each other, while ensuring that the isolation layer 213 has good anti-interference capability, the weight of the wire 21 can be reduced to a certain extent, thereby reducing the weight of the microcatheter 1.

Further, the metal wire has a diameter B, and the copper core has a diameter A, where 1≤A/B≤20.

As such, the copper core and the metal wire have a suitable diameter ratio, which can balance the signal transmission capability and anti-interference capability of the wire 21 while ensuring the appropriate volume of the wire 21, so that the wire 21 can have better comprehensive performance of the wire 21 and thus improve the performance of the microcatheter 1.

In some embodiments, the copper core has a diameter A, and the insulation layer 212 has an outer diameter C, where 0.1≤A/C≤10.

As such, the diameter of the copper core and the outer diameter of the insulation layer 212 have a suitable size ratio, which can balance the signal transmission capability and insulation capability of the wire 21 while ensuring that the wire 21 has a suitable volume, so that the wire 21 has better comprehensive performance and thus improves the performance of the microcatheter 1.

In some embodiments, the intermediate layer 20 is formed in a spiral structure or a mesh structure. Exemplarily, FIG. 2 shows that the intermediate layer 20 is formed in the spiral structure.

Either being formed in the spiral structure, or being formed in the mesh structure, the intermediate layer 20 can meet the support and reinforcement requirements for the microcatheter 1, and can minimize the weight of the microcatheter 1 to a certain extent.

In some embodiments, the microcatheter 1 further includes a coating applied to an inner wall of the inner tube layer 10.

By providing the coating on the inner wall of the inner tube layer 10, the lubrication performance of the inner tube layer 10 can be further improved, thereby improving the smoothness of the microcatheter 1 during substance delivery, and facilitating the delivery of drugs or other minimally invasive intervention devices through the inner tube layer 10.

As a specific embodiment of the present application, the microcatheter 1 includes an inner tube layer 10, an intermediate layer 20, and an outer tube layer 30; the inner tube layer 10 includes a cavity 11 with openings at two ends; the intermediate layer 20 is arranged on the outer wall of the inner tube layer 10 and is formed by winding a plurality of wires 21, and the plurality of wires 21 are adapted for transmitting electrical signals and providing electric energy; the outer tube layer covers the intermediate layer 20 on an outer side thereof; the wire 21 includes a core body 211, an isolation layer 213, and an insulation layer 212; the core body 211 is adapted to transmit electrical signals and/or transmit electric energy; the isolation layer 213 is arranged on an outer side of the core body 211, and the isolation layer 213 is adapted to isolate electrical signals from external signals; the insulation layer 212 is arranged between the core body 211 and the isolation layer 213, and is adapted to insulate and isolate the core body 211 from the isolation layer 213.

According to the embodiment of the present application, the microcatheter 1 includes an inner tube layer 10, an intermediate layer 20, and an outer tube layer 30, the intermediate layer 20 is formed by winding a plurality of wires 21, the plurality of wires 21 are adapted for transmitting electrical signals and transmitting electric energy, and the outer tube layer 30 covers the intermediate layer 20. Thus, the wires 21 for forming the intermediate layer 20 not only can transmit electrical signals, but also has electric energy transmission capability, and thus the microcatheter 1 itself has signal transmission capability and electric energy transmission function, thereby improving the practicality of the microcatheter 1. Moreover, the microcatheter 1 in the embodiment of the present application has a high degree of integration, which can minimize the working diameter of the microcatheter 1 and further expand its application scenarios. When the microcatheter 1 is applied in the minimally invasive intervention apparatus, the functional integration and clinical practicality of the minimally invasive intervention apparatus can be improved, thereby improving the operational convenience of the minimally invasive intervention surgery, reducing difficulty of the surgery, and reducing the burden on medical personnel.

Moreover, by providing the wire 21 to include the core body 211, the isolation layer 213, and the insulation layer 212, while ensuring the signal transmission and electric conductivity of the wire 21, the anti-interference capability of the wire 21 can also be improved, thereby improving the stability of the signal transmission of the wire 21.

The inventors further found that the micro-guide wire cooperating with the microcatheter in existing technologies only has the function of passively guiding the microcatheter and does not have extensible information acquisition and operation functions, mainly because the micro-guide wire lacks electric energy transmission and signal transmission capabilities, and the practicability thereof is relatively limited, which cannot reduce the burden on medical personnel in clinical applications. Thus, intelligent level of vascular intervention surgery is limited to a certain extent, and high skills and rich experience are required for medical personnel. In order to enrich the functions of the micro-guide wire on the basis of its guiding function, so that various sensors and actuators (including cameras, force sensors, ultrasonic generators, etc.) are able to be integrated to the end of the micro-guide wire, it is necessary to innovate the structure of the micro-guide wire, and add anti-interference signal transmission and electric energy transmission functions to the micro-guide wire without increasing the diameter thereof.

In view of the above issues found by the inventors, the inventors improve the structure of the micro-guide wire and propose a micro-guide wire, which includes a functional core and a covering layer; the functional core extends in a first direction and includes a first end, the covering layer covers an outer peripheral surface of a portion of the functional core other than the first end, and the first end can enter a focus area; the functional core includes an inner core and a functional layer, the functional layer covers an outer peripheral surface of the inner core, and the functional layer includes a plurality of line bodies, with at least some of the line bodies capable of transmitting signals and/or electric energy. As such, components for detection can be electrically connected to the line bodies in the micro-guide wire that can transmit signals and/or electric energy, so that the micro-guide wire itself can have signal transmission or electric energy transmission capabilities, thereby enriching the functions of the micro-guide wire and simplifying the process of obtaining current state information of the human body by the micro-guide wire.

Meanwhile, the inventors further propose a minimally invasive intervention apparatus, which includes a detection element, a control component, and the above-mentioned micro-guide wire, the functional layer at the first end of the functional core of the micro-guide wire is electrically connected to the detection element, and an end of the functional layer covered by the covering layer is electrically connected to the control component, that is, the line bodies in the functional layer that can achieve signal transmission and/or electric energy transmission electrically connects the detection element and the control component. In clinical applications, the minimally invasive intervention apparatus in the present application has multiple functions, thereby eliminating the need of functional components for data transmission compared to existing technology, providing convenience for medical personnel in the minimally invasive intervention surgery and reducing work burden.

The embodiments of the present application are described further in detail below in combination with the accompanying drawings.

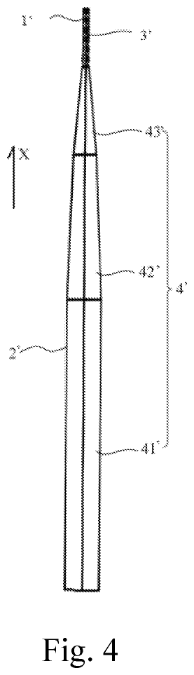

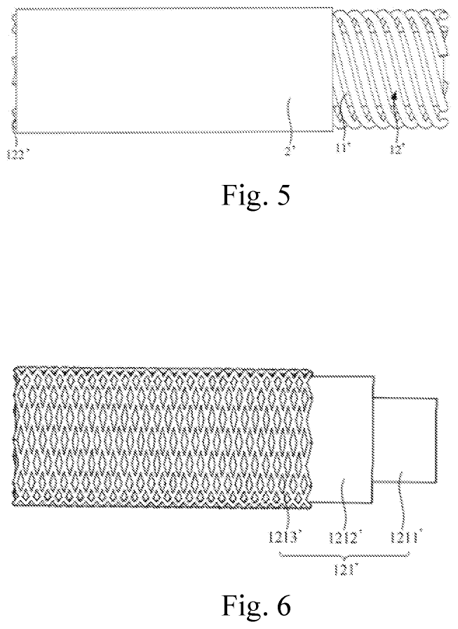

Referring to FIGS. 4 and 5, the embodiment of the present application provides a micro-guide wire, which includes a functional core 1′ and a covering layer 2′; the functional core 1′ extends in a first direction X and includes a first end, the covering layer 2′ covers an outer peripheral surface of a portion of the functional core 1′ other than the first end, and the first end can enter a focus area; the functional core 1′ includes an inner core 11′ and a functional layer, the functional layer covers an outer peripheral surface of the inner core 11′, and the functional layer includes a plurality of line bodies, with at least some of the line bodies capable of transmitting signals and/or electric energy.

It can also be understood that the micro-guide wire includes an inner core 11′, a functional layer 12′, and a covering layer 2′; the inner core 11′ extends in the first direction X, and a movable end of the inner core 11′ is adapted to enter the focus area; the functional layer 12′ is located on an outer side of the inner core 11′, and includes at least one conducting wire 121′, a first end of the conducting wire 121′ is disposed corresponding to the movable end of the inner core 11′, and is adapted to electrically connecting with a certain component; the covering layer 2′ covers a second end of the conducting wire 121′, and in the first direction X, the movable end of the inner core 11′ extends out of the covering layer 2′.

Exemplarily, by adopting the above structure, the micro-guide wire not only has substance delivery function, but also has signal transmission capability. The micro-guide wire can be transformed into various forms according to different usage requirements, for example, it can be curved or bent under external forces.

Optionally, in addition to the conducting wire 121′, the functional layer 12′ can further include a support line, which is cooperatively connected with the conducting wire 121′ to form the functional layer 12′; the conducting wire 121′ may have the same external dimension as the support line, and the support line does not have the function of transmitting signals or electric energy, but only serves to fill the space between the conducting wires and support the conducting wires 121′.

In some available embodiments, the inner core 11′ can be made of stainless steel to transmit torque and provide lateral support; exemplarily, the inner core 11′ can be integrally formed, or can be formed by a plurality of structures being fixed to each other by welding or other manners; for example, the inner core 11′ can be formed by four stainless steel bars, which are fixed together by welding, so as to be connected in sequence in the first direction X to form the inner core 11′.

In the functional layer, the number of conducting wires 121′ is set to 2N, where N is a positive integer, and each conducting wire 121′ is provided to spiral along the first direction X; in a plane perpendicular to the first direction X, a plurality of conducting wires 121′ are uniformly arranged along a circumferential direction of the inner core 11′. Exemplarily, the number of conducting wires 121′ can be 6, where every two conducting wires 121′ are adapted to form a transmission loop 122′, that is, the functional layer includes three transmission loops 122′.

It is easy to understand that the transmission loop 122′ can be provided to achieve different functions, for example, the transmission loop 122′ can be adapted to transmit electric energy to components, or in the transmission loop 122′, one conducting wire 121′ is adapted to transmit signals to the components, and the other conducting wire 121′ is adapted to receive signals from the components to achieve the signal transmission process of the components through the transmission loop 122′.

Optionally, the respective line bodies of the functional layer 12′ can further be connected in a crossing manner to form a mesh structure, so as to further enhance the structural strength of the functional layer 12′.

In some available embodiments, some conducting wires 121′ among the plurality of conducting wires 121′ are adapted to transmit electric energy to the components; some conducting wires 121′ among the plurality of conducting wires 121′ are adapted to transmit signals from the components; the conducting wires 121′ can be implemented in various different forms. For example, some conducting wires 121′ among the plurality of conducting wires 121′ are adapted to transmit signals, while the other conducting wires 121′ are adapted to transmit electric energy. Alternatively, some conducting wires 121′ among the plurality of conducting wires 121 are adapted to transmit signals, some other conducting wires 121′ are adapted to transmit electric energy, and the remaining conducting wires 121′ are only adapted to support and reinforce the micro-guide wire. Alternatively, while transmitting signals and/or electric energy, all the conducting wires 121′ can support a wall of the covering layer of the micro-guide wire to maintain the shape thereof, avoid the inner core 11′ from being compressed, and also can transmit forces and moments generated by the surgeon's hand operation during the minimally invasive surgery. The embodiments of the present application do not further limit this aspect.

It is worth mentioning that some of the plurality of conducting wires 121′ can have both signal transmission and electric energy transmission functions. As a result, the plurality of conducting wires 121′ can not only transmit signals, but also can transmit electric energy, thereby further improving the functional practicality and integration of micro-guide wire.

Referring to FIG. 6, the conducting wire 121′ includes a core line 1211′, an insulation layer 1212′, and a shielding layer 1213′, where the core line 1211′ can be adjusted according to actual situations, and for example, the core line 1211′ can be provided to be a structure such as an optical fiber or a conductive core, so that the core line 1211′ can be adapted to transmit signals and/or transmit electric energy to components; for example, the core line 1211′ can be made of nickel titanium alloy or other alloy materials; the core line 1211′ may have a diameter of less than 1 millimeter, and for example, 30 micrometers can be selected; the insulation layer 1212′ can be made of PTFE (Poly tetra fluoroethylene) or other insulation materials, the insulation layer 1212′ is arranged on an outer side of the core line 1211′ so as to insulate and isolate the core line 1211′ from the shielding layer 1213′; the insulation layer 1212′ may have a thickness of 50 microns, and a ratio between the thickness of the insulation layer 1212′ and that of the core line 1211′ is in a range of 0.05 to 5. The shielding layer 1213′ is arranged on an outer side of the insulation layer 1212′ and is adapted to isolate signals from external signals, and exemplarily, the shielding layer 1213′ can be made of silver or other materials with good shielding function, such as other metal materials with high conductivity; the shielding layer 1213′ includes a plurality of metal wires, which are arranged in a crossing manner; the shielding layer 1213′ can have a thickness of 30 microns to ensure that the thickness is sufficient to protect the functional core 1; a ratio between the thickness of the shielding layer 1213′ and the diameter of the core line 1211′ is in a range of 0.01 to 10.

Moreover, by adopting the above configuration, the conducting wire 121′ includes the core line 1211′, the shielding layer 1213′, and the insulation layer 1212′, while ensuring that the conducting wire 121′ has signal transmission and conductivity without increasing the diameter of the micro-guide wire, the anti-interference capability of the conducting wire 121′ can also be improved, thereby improving the stability of signal transmission of the conducting wire 121′.

In some possible embodiments, the covering layer 2′ can be made of TPU (Thermoplastic polyurethane) elastomer rubber, so as to ensure good elasticity thereof.

Referring to FIG. 4, the micro-guide wire includes an end portion 3′ and an extension portion 4′, the first end of the functional core 1′ that is not covered by the covering layer 2′ forms the end portion 3′, the portion of the functional core 1′ that is covered by the covering layer 2′ and the covering layer 2′ form the extension portion 4′. The extension portion 4′ can include a support portion 41′, a transition portion 42′, and a guide portion 43′ connected to each other. In a cross-section taken along a plane perpendicular to the first direction X, radial dimensions of the transition portion 42′ and the guide portion 43′ gradually decrease in a direction approaching the movable end of the inner core 11′.

Optionally, according to actual surgical requirements, in various surgical instruments, the extension portion 4′ can include merely the support portion 41′, include merely the support portion 41′ and the transition portion 42′, include merely the support portion 41′ and the guide portion 43′, or include all of the support portion 41′, the transition portion 42′, and the guide portion 43′.

Exemplarily, the support portion 41′ is formed as a cylindrical support portion 41′, and the cylindrical support portion 41′ is disposed at one end of the transition portion 42′ away from the movable end of the inner core 11′; in the first direction X, a length of the cylindrical support portion 41′ can be adjusted according to actual situations, and for example, a length of the micro-guide wire is set to 1 meter to 5 meters, optionally 2 meters, a length of the covering layer 2′ can be 1.8 meters, the length of the cylindrical support portion 41′ can be 1 meter, a ratio between the length of the covering layer 2′ and the length of the micro-guide wire is in a range of 0.01 to 0.5, and a ratio between the length of the cylindrical support portion 41′ and the length of the micro-guide wire is in a range of 0.01 to 1.

The transition portion 42′ is formed as a circular truncated cone shaped transition portion 42′, and by taking a cross-section along a plane perpendicular to the first direction X, a cross-sectional area of the circular truncated cone shaped transition portion 42′ gradually decreases in a direction approaching the movable end of the inner core 11′; in the first direction X, a length of the circular truncated cone shaped transition portion 42′ can be selected as 0.5 meters, a diameter of a cross-section of the circular truncated cone shaped transition portion 42′ connected to the cylindrical support portion 41′ can be selected as 100 micrometers, a diameter of a cross-section of the circular truncated cone shaped transition portion 42′ connected to the guide portion 43′ can be selected as 50 micrometers, and a ratio between the length of the circular truncated cone shaped transition portion 42′ and the length of the micro-guide wire is in a range of 0.01 to 0.7; a ratio between a cross-sectional diameter of the circular truncated cone shaped transition portion 42′ and a cross-sectional diameter of the micro-guide wire is in a range of 0.1 to 1; two ends of the circular truncated cone shaped transition portion 42′ are respectively connected to the cylindrical support portion 41′ and the guide portion 43′, and cross-sections of connected two parts at each connection are consistent in shape and size.

The guide portion 43′ is connected to one end of the circular truncated cone shaped transition portion 42′ away from the cylindrical support portion 41′, and in the first direction X, a length of the guide portion 43′ is smaller than the length of the circular truncated cone shaped transition portion 42′, and the length of the guide portion 43′ can be set to 30 microns; a diameter of a cross-section of the guide portion 43′ connected to the circular truncated cone shaped transition portion 42′ can be 50 microns, and a diameter of a cross-section of the guide portion 43′ away from the circular truncated cone shaped transition portion 42′ can be 30 microns, so that an end of the guide portion 43′ away from the circular truncated cone shaped transition portion 42′ is smoothly connected to the functional core 1′. A ratio between the length of the guide portion and the length of the micro-guide wire is in a range of 0.01 to 0.5, a ratio between the cross-sectional diameter of an end of the guide portion connected to the circular truncated cone shaped transition portion and the cross-sectional diameter of the micro-guide wire is in a range of 0.1 to 1, and it is available as long as the cross-sectional diameter of the end of the guide portion 43′ away from the circular truncated cone shaped transition portion 42′ is smaller than the cross-sectional diameter of the end of the guide portion 43′ connected to the circular truncate cone shaped transition portion 42′.

In summary, the micro-guide wire according to the embodiment of the present application includes an inner core, a functional layer and a covering layer; the inner core extends in the first direction, and the movable end of the inner core is adapted to enter the focus area; the functional layer is arranged on the outer side of the inner core, and includes at least one conducting wire; a first end of the conducting wire is disposed corresponding to the movable end of the inner core, and is adapted for being electrically connected with components; the covering layer covers a second end of the conducting wire, and in the first direction, the movable end of the inner core extends out of the covering layer. Thus, when the movable end of the inner core of the micro-guide wire delivers drugs or micro-instruments into the focus area cooperating with the microcatherter, the first end of the conducting wires can carry the components into the focus area to obtain the current state information of the micro-guide wire or the human body through the components; thus, by electrically connecting with the components by the conducting wires of the micro-guide wire, the function of the micro-guide wire can be enriched, and the process of obtaining the current state information of the micro-guide wire or the human body can be simplified, thereby effectively improving the intelligence level of vascular intervention surgery, and reducing the requirements for medical personnel's skill and experience; in the case that the diameter of the micro-guide wire is not increased, the functions of the micro-guide wire are enriched on the basis of the guiding function, so that the end of the micro-guide wire can be integrated with various sensors and actuators (including cameras, force sensors, ultrasound generators, etc.), and also the anti-interference signal transmission and electric energy transmission functions can be enhanced.

The embodiment of the present application further provides a minimally invasive intervention apparatus, which includes a detection element, a control component, and the aforementioned micro-guide wire, the functional layer at the first end of the functional core is electrically connected to the detection element, and the end of the functional layer covered by the covering layer is electrically connected to the control component. Specifically, the line bodies in the functional layer with signal transmission and/or electric energy transmission functions can realize the electrical connection between the detection element and the control component.

For example, the control component can be connected to the transmission loop of the micro-guide wire, so that the signal transmission process of the detection element and the process of transmitting electric energy to the detection element can be achieved through the transmission loop.

In some available embodiments, the control component can emit and receive signals, and the control component can be an independent device or a collection of multiple connected devices. The control component can also have control functions, such as being a controller with signal emission and reception capabilities. The control component itself can further have display functions, such as being configured with display panels, or the control component can be connected to external display devices. The control component can be provided with a power source, such as batteries, or the control component can be connected to external power devices.

The detection element can be of various structures, such as a probe with force sensing function, a camera with image sensing function, or a micro robot with surgical operation function. The detection components can be one or more, and in case of more than one detection elements, the functions of the more than one detection elements can be the same or different. Alternatively, one detection element can be integrated with multiple functions simultaneously, which can further enhance the versatility of the minimally invasive intervention apparatus in clinical applications and thus enhance the practicality thereof. In clinical applications, the detection components can be configured with various functional modes according to different application scenarios of the minimally invasive intervention surgery, and the present application does not limit this.

It should be noted that the signal referred to in the embodiment of the present application can be either an electrical signal or an optical signal.

The minimally invasive intervention apparatus including the micro-guide wire of the present application has multifunctional applications and can replace some auxiliary medical means in minimally invasive intervention surgery in clinical applications, providing convenience for operation of the minimally invasive intervention surgery, reducing surgical difficulty, and reducing the burden on medical personnel. Moreover, the micro-guide wire of the minimally invasive intervention apparatus in the present application has high degree of integration, can minimize the working diameter of the micro-guide wire and expand the application scenarios of the minimally invasive intervention apparatus.

Ordinary skilled in this field can understand that the microcatheter in the embodiments of the present application can be used in cooperation with any micro-guide wire in the prior art. The micro-guide wire in the embodiments of the present application can be used in cooperation with any microcatheter in the prior art, and the microcatheter and micro-guide wire in the embodiments of the present application can also be used in cooperation with each other.

Although the present disclosure has been described with reference to preferred embodiments, various modifications can be made thereto and components therein can be replaced with equivalents without departing from the scope of the present disclosure. Especially, as long as there is no structural conflict, various technical features mentioned in various embodiments can be combined in any manner. The present disclosure is not limited to the specific embodiments disclosed herein, but includes all technical solutions falling within the scope of the claims.

What is claimed is:

1. A minimally invasive intervention device, comprising:
an inner layer, an intermediate layer, and an outer layer,
wherein the intermediate layer covers an outer peripheral surface of the inner layer, and the outer layer covers an outer peripheral surface of the intermediate layer; and
the intermediate layer comprises a plurality of line bodies, at least some of the line bodies can transmit signals and/or electric energy.

2. The minimally invasive intervention device according to claim 1, wherein the plurality of line bodies are formed as a plurality of conducting wires, and the plurality of conducting wires are adapted for transmitting signals and/or transmitting electric energy.

3. The minimally invasive intervention device according to claim 2, wherein each of the conducting wires comprises:
a core body adapted for transmitting signals and/or transmitting electric energy;
an isolation layer arranged on an outer side of the core body, and adapted to isolate the signals from external signals; and
an insulation layer arranged between the core body and the isolation layer, and adapted to insulate and isolate the core body from the isolation layer.

4. The minimally invasive intervention device according to claim 3, wherein the isolation layer comprises a plurality of metal wires arranged in a crossing manner.

5. The minimally invasive intervention device according to claim 4, wherein the core body is made of metal or alloy material, the isolation layer is made of conductive material, and the insulation layer is made of insulating material.

6. The minimally invasive intervention device according to claim 5, wherein the core body is formed as a copper core, the copper core has a diameter A, and the metal wire has a diameter B, wherein 1≤A/B≤20.

7. The minimally invasive intervention device according to claim 5, wherein the core body is formed as a copper core, the copper core has a diameter A, and the insulation layer has an outer diameter C, wherein 0.1≤A/C≤10.

8. The minimally invasive intervention device according to claim 1, wherein the inner layer is formed as an inner tube layer, which comprises a cavity with openings at two ends, the intermediate layer is formed by winding the plurality of line bodies, and the intermediate layer is formed as a spiral or mesh structure.

9. The minimally invasive intervention device according to claim 8, further comprising a coating coated on an inner wall of the inner tube layer.

10. The minimally invasive intervention device according to claim 1, wherein the inner layer is formed as an inner core, and the inner core and the intermediate layer form a functional core; and
the functional core extends in a first direction and comprises a first end, the outer layer covers an outer peripheral surface of a portion of the functional core other than the first end, and the first end can enter a focus area.

11. The minimally invasive intervention device according to claim 10, wherein each line body is arranged spirally around an outer peripheral surface of the inner core, and in a cross-section perpendicular to the first direction, the plurality of line bodies are arranged along a circumferential direction of the inner core;
or, the respective line bodies are crossed by each other to form a mesh structure.

12. The minimally invasive intervention device according to claim 10, wherein the line bodies comprise a plurality of support lines and conducting wires with the number of 2N, N being a positive integer, the conducting wires can transmit signals and/or electric energy, and each conducting wire forms a transmission loop with another conducting wire.

13. The minimally invasive intervention device according to claim 12, wherein the intermediate layer is formed with a plurality of transmission loops, some of the transmission loops are adapted for transmitting electric energy, and others of the transmission loops are adapted for transmitting signals.

14. The minimally invasive intervention device according to claim 10, comprising an end portion and an extension portion, wherein a portion of the functional core not covered by the outer layer forms the end portion, and the portion of the functional core covered by the outer layer and the outer layer form the extension portion; in a cross-section perpendicular to the first direction, a radial dimension of the extension portion gradually decreases in a direction from the extension portion to the end portion.

15. The minimally invasive intervention device according to claim 14, wherein the extension portion comprises a support portion, a transition portion and a guide portion connected to each other in the direction from the extension portion to the end portion;
the support portion is formed in a cylindrical shape; and
radial dimensions of the transition portion and the guide portion both gradually decrease in the direction from the extension portion to the end portion.

16. A minimally invasive intervention apparatus, comprising:
the minimally invasive intervention device as claimed in claim 1;
a signal transceiver/power supply connected to one end of the minimally invasive intervention device; and
an actuator/sensor connected to an end of the minimally invasive intervention device away from the signal transceiver/power supply.

17. A minimally invasive intervention apparatus, comprising a sensing/executing element, a control/computing/power supply element, and the minimally invasive intervention device as claimed in claim 10, the intermediate layer of the minimally invasive intervention device is electrically connected to the sensing/executing element at a first end of the line bodies, and an end of the intermediate layer covered by the outer layer is electrically connected to the control/computing/power supply element.
